(19) United States
(12) Patent Application Publication
(10) Pub. No.: US 2025/0035632 A1
(43) Pub. Date: Jan. 30, 2025

(54) SORD AS BIOMARKER FOR PREDICTING PROGNOSIS AFTER LIVER CANCER SURGERY

(71) Applicants: INSTITUT PASTEUR KOREA, Seongnam-si, Gyeonggi-do (KR); THE ASAN FOUNDATION, Seoul (KR); UNIVERSITY OF ULSAN FOUNDATION FOR INDUSTRY COOPERATION, Ulsan (KR)

(72) Inventors: Kang Mo KIM, Seoul (KR); Haeng Ran SEO, Seoul (KR); Su-Yeon LEE, Seongnam-si, Gyeonggi-do (KR); Jin-Sun KIM, Seongnam-si, Gyeonggi-do (KR); Yeonhwa SONG, Seoul (KR); Sanghwa KIM, Suwon-si, Gyeonggi-do (KR)

(73) Assignees: INSTITUT PASTEUR KOREA, Seongnam-si, Gyeonggi-do (KR); THE ASAN FOUNDATION, Seoul (KR); UNIVERSITY OF ULSAN FOUNDATION FOR INDUSTRY COOPERATION, Ulsan (KR)

(21) Appl. No.: 18/707,174

(22) PCT Filed: Nov. 11, 2022
(86) PCT No.: PCT/KR2022/017789

§ 371 Date: May 3, 2024

(30) Foreign Application Priority Data
Nov. 11, 2021 (KR) 10-2021-0155200

(51) Int. Cl. G01N 33/574

(52) U.S. Cl. CPC G01N 33/57438; G01N 2333/904

(57) ABSTRACT

The present invention relates to a method, a composition, and a kit for preoperative prediction of prognosis after liver cancer surgery at an early stage by detecting, in a blood sample obtained from a liver cancer patient prior to surgery, an SORD marker, or an SORD marker together with an AFP marker.

### TECHNICAL FIELD

This application claims the benefit of Korean Patent Application No. 10-2021-0155200 filed on Nov. 11, 2021, and the entire disclosure of the specification and drawings thereof is incorporated herein by reference.

The present disclosure relates to a biomarker for predicting a prognosis after liver cancer surgery.

### BACKGROUND ART

Recent biomarker discovery processes have shown a tendency to increase development cost while decreasing a success rate. This is partly due to the disparity between basic research and clinical research. Basic researchers use cell lines to conduct experiments for convenience in the experiments, but the phenomena that occur in cell lines bring significant difference from responses shown at an individual level.

In order to solve these problems, 3D culture systems to cultivate cell lines similar to the environment of an individual are gaining popularity and emerging as a way to narrow a gap between basic research and clinical research.

Unlike single-layer culture (2D culture, where cultured cells are spread out in a single layer on a medium), 3D culture capable of culturing cells under a 3D environment similar to in vivo has been reported in many papers that specific functions of cells and tissues are maintained for a long time with relatively high efficiency, and, in the comparison of sensitivity to toxic drugs between 2D culture, 3D culture, and in vivo system, it is reported in the paper that the outcome of 3D culture is surprisingly similar to the in vivo system.

On the other hand, liver cancer or hepatocellular carcinoma (HCC) is the most common type of cancer in adults and known to be one of cancers with an extremely poor prognosis, with death within 1 year in the surgically unresectable case while recurrence is observed in more than 50% of cases within 5 years even after surgical resection. Considering these clinical realities, technology that enables prediction of cancer prognosis at an early stage is the most realistic alternative to cancer treatment and a guideline for the next generation of liver cancer care.

Currently, the most well-known biomarkers for liver cancer-related diagnosis, prognosis determination, and treatment evaluation are AFP and protein induced by vitamin K absence (PIVKA)-II, which are not satisfactory in terms of specificity and sensitivity. The usefulness of blood alpha-fetoprotein (AFP) in the diagnosis of hepatocellular carcinoma is well known. In addition to the diagnosis of advanced hepatocellular carcinoma, regular AFP measurement is necessary for early detection because hepatocellular carcinoma develops in 3-10% of patients with cirrhosis every year in the natural course. However, AFP is elevated at high concentration not only in hepatocellular carcinoma, but also in benign diseases such as alcoholic hepatitis, chronic hepatitis, or cirrhosis, causing many false positives with the actual positivity rate of 50-60% only. PIVKA-II is des-r-carboxyprothrombin (DCP), that is, non-coagulant abnormal prothrombin, with report to have sensitivity and specificity of 48.2% and 95.9%, respectively, in the diagnosis of hepatocellular carcinoma, independent of serum AFP. These biological markers are currently applied in the clinical field, but they do not reflect biological features of all liver cancers.

In addition, biomarker tests capable of accurately predicting the prognosis before hepatocellular carcinoma surgery have not yet been developed, and even if they have any significance as a prognostic factor, they are not yet used as screening tests due to poor accuracy.

A number of documents are referenced, and citations are indicated throughout the specification. The disclosure of the cited documents is incorporated herein by reference in its entirety, and the level of the technical field to which the present disclosure pertains and the content of the present disclosure are more clearly explained.

### DISCLOSURE OF THE INVENTION

### Technical Goals

The inventors have sought to develop a method capable of early diagnosing a prognosis of liver cancer, especially a molecular diagnostic method capable of preoperatively diagnosing postoperative survival or probability of recurrence of liver cancer patients. As a result, the inventors completed the present disclosure by determining that an L-iditol-2 dehydrogenase (SORD) protein in a blood sample is closely related to the prognosis after liver cancer surgery to be used to predict the postoperative prognosis prior to surgery.

Therefore, an object of the present disclosure is to provide a method of detecting a SORD marker from a blood sample to preoperatively diagnose the prognosis of liver cancer surgery.

Another object of the present disclosure is to provide a composition for early diagnosing a prognosis of liver cancer surgery by detecting a SORD marker from a blood sample.

Another object of the present disclosure is to provide a kit for early diagnosing a prognosis of liver cancer by detecting a SORD marker from a blood sample.

More specifically, the present disclosure provides the following example embodiments:

Example Embodiment 1. A method of detecting an L-iditol-2 dehydrogenase (SORD) marker from a blood sample obtained from a liver cancer patient in order to provide information necessary for diagnosing a prognosis after liver cancer surgery.

Example Embodiment 2. The method of Example Embodiment 1, wherein the information is provided prior to liver cancer surgery.

Example Embodiment 3. The method according to any one of the preceding Example Embodiments, wherein the blood sample is serum.

Example Embodiment 4. The method according to any one of the preceding Example Embodiments,

wherein the method comprises:
detecting a level of SORD included in serum of the liver cancer patient prior to liver cancer surgery; and
determining whether the serum SORD level is greater than or equal to 15 ng/mL.

Example Embodiment 5. The method according to any one of the preceding Example Embodiments, wherein, when the serum SORD level is greater than or equal to 15 ng/mL, information for diagnosing a poor prognosis after liver cancer surgery is provided.

Example Embodiment 6. The method according to any one of the preceding Example Embodiments,

wherein the method further comprises:
detecting a level of alpha-fetoprotein (AFP) included in serum of the liver cancer patient prior to liver cancer surgery; and
determining whether the serum AFP level is greater than or equal to 400 ng/mL.

Example Embodiment 7. The method according to any one of the preceding Example Embodiments, wherein, when the serum SORD level is greater than or equal to 15 ng/mL, and the serum AFP level is greater than or equal to 400 ng/mL, information for predicting a poor prognosis after liver cancer surgery is provided.

Example Embodiment 8. The method according to any one of the preceding Example Embodiments, wherein detection of the SORD or AFP level is performed by immunoassay or immunostaining.

Example Embodiment 9. The method according to any one of the preceding Example Embodiments, wherein the immunoassay or immunostaining is performed using an antibody specifically binding to SORD or AFP.

Example Embodiment 10. A composition for early diagnosing a prognosis of liver cancer surgery to detect a SORD marker from a blood sample, including an antibody specifically binding to an L-iditol-2 dehydrogenase (SORD) protein.

Example Embodiment 11. The composition according to any one of the preceding Example Embodiments, wherein the composition further includes an antibody specifically binding to an alpha-fetoprotein (AFP) protein.

Example Embodiment 12. The composition according to any one of the preceding Example Embodiments, wherein the composition is a composition for detecting a serum protein.

Example Embodiment 13. A kit for early diagnosing a prognosis of liver cancer surgery to detect a SORD marker from a blood sample, including an antibody specifically binding to an L-iditol-2 dehydrogenase (SORD) protein.

Example Embodiment 14. The kit according to any one of the preceding Example Embodiments, wherein the kit further includes an antibody specifically binding to an alpha-fetoprotein (AFP) protein.

Example Embodiment 15. The kit according to any one of the preceding Example Embodiments, wherein the blood sample is serum, and the kit includes a manual that instructs to provide information for predicting a poor prognosis after liver cancer surgery, when a serum SORD level is greater than or equal to 15 ng/mL; or when the serum SORD level is greater than or equal to 15 ng/mL and a serum AFP level is greater than or equal to 400 ng/mL.

Other objects and advantages of the present disclosure are made clearer by the detailed description, claims, and drawings of the disclosure below.

### Technical Solutions

One aspect of the present disclosure is to provide a method of detecting an L-iditol-2 dehydrogenase (SORD) marker from a blood sample obtained from liver cancer patients in order to provide information necessary for diagnosing a prognosis after liver cancer surgery at an early stage, preferably prior to liver cancer surgery.

Hepatocellular carcinoma (HCC) is the most common type of primary liver cancer with a high prevalence and incidence in Asia. Hepatectomy is a treatment of choice for patients with early-stage or resectable hepatocellular carcinoma, but the results are unsatisfactory. Due to the high recurrence rate, the survival of these patients remains low. The 5-year recurrence rate was 68% in patients with single hepatocellular carcinoma (2 cm) after hepatectomy, and recurrence of hepatocellular carcinoma adversely affects the long-term survival of patients. Therefore, predicting recurrence after resection for hepatocellular carcinoma is important for selecting an appropriate surgical candidate. In previous studies, it was noted that preoperative serum alpha-fetoprotein (AFP) levels as well as various histological features of the tumor, such as tumor size and microvascular invasion, were independent predictors of recurrence after resection. However, histological features are limited because they cannot be preoperatively assessed. In addition, AFP has relatively low sensitivity and specificity in accurately predicting hepatocellular carcinoma (Tateishi, R. et al., Diagnostic accuracy of tumor markers for hepatocellular carcinoma: a systematic review, Hepatology international, 2008(2):17-30), and association between AFP and surgical outcomes is still controversial (Blank, S. et al., Assessing prognostic significance of preoperative alpha-fetoprotein in hepatitis B-associated hepatocellular carcinoma: normal is not the new normal. Ann Surg Oncol 2014(21):986-994; Shim, J. H. et al., Is serum alpha-fetoprotein useful for predicting recurrence and mortality specific to hepatocellular carcinoma after hepatectomy? A test based on propensity scores and competing risks analysis. Ann Surg Oncol 2012(19):3687-3696). Therefore, new prognostic markers are still needed to predict outcomes in patients with hepatocellular carcinoma after resection.

Inflammation, necrosis, and liver regeneration induced by various liver diseases play an important role in promoting development of hepatocellular carcinoma. More than 90% of hepatocellular carcinomas occur in association with liver damage and inflammation, making them a definite example of inflammation-related cancers. Sorbitol dehydrogenase (SORD), an enzyme in the polyol pathway that converts sorbitol to fructose, reflects liver damage. SORD, similar to alanine aminotransferase (ALT), is predominant in the liver.

An object of the present disclosure is to determine whether a level of sorbitol dehydrogenase (SORD), an enzyme that reflects liver damage, is related with a recurrence-free survival (RFS) period. The discovery of the present disclosure, in that SORD level ≥15 ng/mL is related with the shorter recurrence-free survival, may be helpful in determining which patients are more suitable for surgery in HCC.

In one example embodiment, a method of the present disclosure may be an in-vitro method. The method of the present disclosure may include detecting a level of SORD included in serum of a liver cancer patient prior to liver cancer surgery; and determining whether the serum SORD level is greater than or equal to 15 ng/mL, wherein, when the serum SORD level is greater than or equal to 15 ng/mL, information for diagnosing a poor prognosis after liver cancer surgery may be provided.

In particular, in a preferred example embodiment, serum AFP and SORD levels are used as two independent prognostic factors for RFS. Specifically, the method further includes detecting a level of alpha-fetoprotein (AFP) included in serum of liver cancer patients prior to liver cancer surgery; and determining whether the serum AFP level is greater than or equal to 400 ng/mL.

When patients were stratified by preoperative serum SORD and/or AFP levels, if the serum SORD level is greater than or equal to 15 ng/mL, information for diagnosing a poor prognosis after liver cancer surgery is provided, and patients with serum SORD levels greater than or equal to 15 ng/mL and serum AFP levels greater than or equal to 400 ng/mL had a markedly poor prognosis with the lowest RFS rate.

As such, preoperative serum SORD is an effective prognostic factor for post-resection hepatocellular carcinoma, and it is expected to provide clinical assistance in selecting patients suitable for surgery, especially when checking such as serum AFP levels.

In the prognosis diagnostic method of the present disclosure, analysis of expression levels of SORD and/or AFP proteins may be carried out through a variety of methods known in the art. Preferably, the analysis may be carried out according to an immunoassay or immunostaining protocol.

The immunoassay or immunostaining formats include, but are not limited to, radioimmunoassay, immunoprecipitation, enzyme-linked immunosorbent assay (ELISA), capture-ELISA, inhibition or competition assays, sandwich assays, flow cytometry, immunofluorescence staining, and immunoaffinity purification. The method of immunoassay or immunostaining is described in Enzyme Immunoassay, E. T. Maggio, ed., CRC Press, Boca Raton, Florida, 1980; Gaastra, W., Enzyme-linked immunosorbent assay (ELISA), in Methods in Molecular Biology, Vol. 1, Walker, J. M. ed., Humana Press, NJ, 1984; and Ed Harlow and David Lane, Using Antibodies: A Laboratory Manual, Cold Spring Harbor Laboratory Press, 1999, of which are incorporated herein by reference.

For example, if the method of the present disclosure is carried out in accordance with the radioimmunoassay method, antibodies (antibodies specifically binding to the marker) labeled with a radioisotope (e.g., C14, I125, P32, and S35) may be used.

If the method of the present disclosure is carried out in an ELISA manner, a specific example embodiment of the present disclosure may include (i) coating a surface of a solid substrate with an unknown sample to be analyzed; (ii) reacting the sample with the marker-specific antibody as a primary antibody; (iii) reacting the resulting product of step (ii) with an enzyme-bound secondary antibody; and (iv) measuring an activity of the enzyme.

Hydrocarbon polymers (such as polystyrene and polypropylene), glass, metal or gel, or microtiter plates may be suitable as the solid substrate.

Enzymes bound to the secondary antibody include, but are not limited to, enzymes that catalyze chromogenic reactions, fluorescence reactions, luminescence reactions, or infrared reactions, including, for example, alkaline phosphatase, β-galactosidase, horse radish peroxidase, luciferase, and cytochrome P450. When alkaline phosphatase is used as an enzyme that binds to the secondary antibody, chromogenic substrates such as bromochloroindolyl phosphate (BCIP), nitro-blue tetrazolium (NBT), naphthol-AS-B1-phosphate, and enhanced chemifluorescence (ECF) may be used as a substrate, and if horse radical peroxidase is used, substrates such as chloronaphthol, aminoethyl carbazole, diaminobenzidine, D-luciferin, lucigenin(bis-N-methylacridinium nitrate), resorufin benzyl ether, luminol, Amplex Red reagent (10-acetyl-3,7-dihydroxyphenoxazine), p-phenylenediamine-HCl and pyrocatechol (HYR), tetramethylbenzidine (TMB), 2,2′-azine-di[3-ethylbenzthiazoline sulfonate] (ABTS), o-phenylenediamine (OPD) and naphtol/pyronine, glucose oxidase and nitroblue tetrazolium (t-NBT), and phenazine methosulfate (m-PMS) may be used.

If the method of the present disclosure is carried out in a capture-ELISA manner, a specific example embodiment of the present disclosure includes: (i) coating a surface of a solid substrate with antibodies against a molecular marker as a capturing antibody; (ii) reacting a sample with the capture antibody; (iii) reacting a resulting product of step (ii) with a detecting antibody against the molecular marker that is bound with a label to generate a signal; and (iv) measuring the signal generated from the label.

The detection antibody has a label that generates a detectable signal, wherein the label includes, but not limited to, chemicals (e.g., biotin), enzymes (e.g., alkaline phosphatase, β-galactosidase, horse radish peroxidase, and cytochrome P450), radioactive substances (e.g., C14, I125, P32, and S35), fluorescent substances (e.g., fluorescein), luminescent substances, chemiluminescent, and fluorescence resonance energy transfer (FRET). Various labels and labeling methods are described in Ed Harlow and David Lane, Using Antibodies: A Laboratory Manual, Cold Spring Harbor Laboratory Press, 1999.

In the ELISA method and the capture-ELISA method, the final enzyme activity measurement or signal measurement may be carried out according to the various methods known in the art. If biotin is used as a label, the signal may be easily detected with streptavidin, and if luciferase is used, with luciferin.

On the other hand, the method of the present disclosure may be carried out by conventional flow cytometry (Ormerod, M. G., ed. 1990, Flow Cytometry: A Practical Approach. IRL Press), magnetic-activated cell sorting (MACS; Robert David, et al., Stem Cells, 23:477-482(2005), or immunoaffinity purification (Ed Harlow and David Lane, Using Antibodies: A Laboratory Manual, Cold Spring Harbor Laboratory Press, 1999).

Antibodies specifically binding to the molecular markers may be prepared by methods conventionally conducted in the art, such as the fusion method (Kohler and Milstein, European Journal of Immunology, 6:511-519(1976)), the recombinant DNA method (U.S. Pat. No. 4,816,567), or the phage antibody library method (Clackson et al, Nature, 352:624-628(1991) and Marks et al, J. Mol. Biol., 222:58, 1-597(1991)). General processes for antibody preparation are described in detail in Harlow, E. and Lane, D., Using Antibodies: A Laboratory Manual, Cold Spring Harbor Press, New York, 1999; Zola, H., Monoclonal Antibodies: A Manual of Techniques, CRC Press, Inc., Boca Raton, Florida, 1984; and Coligan, CURRENT PROTOCOLS IN IMMUNOLOGY, Wiley/Greene, NY, 1991, of which are incorporated herein by reference.

By analyzing the intensity of a final signal by the immunoassay process described above, it is possible to predict the prognosis after liver cancer surgery.

Another aspect of the present disclosure is to provide a composition for early diagnosing a prognosis of liver cancer surgery to detect a SORD marker from a blood sample, which includes an antibody specifically binding to an L-iditol-2 dehydrogenase (SORD) protein.

In a preferred example embodiment, the composition for early diagnosing a prognosis of liver cancer surgery may further include an antibody specifically binding to an alpha-fetoprotein (AFP) protein and may be a composition for detecting a serum protein.

Another aspect of the present disclosure is to provide a kit for early diagnosing a prognosis of liver cancer surgery to detect a SORD marker from a blood sample, which includes an antibody specifically binding to an L-iditol-2 dehydrogenase (SORD) protein.

In a preferred example embodiment, the kit for early diagnosing a prognosis of liver cancer surgery may further include an antibody specifically binding to an alpha-fetoprotein (AFP) protein.

The kit may be a kit for detecting a serum protein using serum as a blood sample, wherein when a serum SORD level is greater than or equal to 15 ng/mL; or preferably, when the serum SORD level is greater than or equal to 15 ng/mL and a serum AFP level is greater than or equal to 400 ng/mL, the kit may include a manual that instructs to provide information for diagnosing a poor prognosis after liver cancer surgery.

If the kit for early diagnosing a prognosis of liver cancer surgery of the present disclosure is applied to immunoassay, the kit of the present disclosure may optionally include a secondary antibody and a substrate of a label. In addition, the kit of the present disclosure may be prepared in a number of separate packaging or compartments including reagent components described above.

### Advantageous Effects

The present disclosure provides a technology for predicting a postoperative prognosis prior to liver cancer surgery by detecting a SORD marker, preferably along with an AFP marker, in a blood sample obtained from a liver cancer patient. Therefore, the present disclosure facilitates decision-making on suitable surgical targets to avoid unnecessary surgery in patients who are expected to have a low survival rate.

### BRIEF DESCRIPTION OF DRAWINGS

FIG. 1A shows a Kaplan-Meier plot for recurrence-free survival (RFS) of patients with hepatocellular carcinoma after curative resection, indicating that the patients are stratified into two groups according to preoperative serum SORD levels (<15 ng/mL and ≥15 ng/mL).

FIG. 1B shows a Kaplan-Meier plot for recurrence-free survival (RFS) of patients with hepatocellular carcinoma after curative resection, indicating that the patients are stratified into four groups according to preoperative serum SORD levels (<5, 5-10, 10-15, ≥15 ng/mL).

FIG. 2 shows a forest plot of recurrence-free survival by preoperative serum SORD levels in various subgroups of patients, indicating a hazard ratio of patients with elevated serum SORD levels (≥15 ng/mL) compared to patients with low serum SORD levels (<15 ng/mL).

FIG. 3 shows a Kaplan-Meier plot for recurrence-free survival stratified by serum levels of AFP and SORD, indicating a hazard ratio adjusted for sex and indocyanine green retention rate at 15 minutes (ICG R15).

### MODES FOR CARRYING OUT THE INVENTION

Hereinafter, the present disclosure will be described in more detail through example embodiments. These example embodiments are merely intended to describe the present disclosure more specifically, and it will be apparent to those skilled in the art to which the present disclosure pertains that the scope of the present disclosure is not limited by these example embodiments.

### Example

### I. Materials and Methods

### 1. Study Design and Population

Records of 150 patients who underwent hepatectomy for hepatocellular carcinoma at Asan Medical Center in Seoul from January 2011 to December 2012 and had peripheral blood stored in the Bio-Resource Center of Asan Medical Center were randomized and included in the analysis. All patients signed an informed consent form to use this information in future studies. Exclusion criteria consisted of (a) patients who underwent liver transplantation, (b) patients whose tumors were incompletely resected, and (c) patients classified as Barcelona Clinic Liver Cancer (BCLC) stage B or C. Patients with these BCLCs stage B or C were later included in the expansion analysis. Ultimately, 92 patients were included in the primary analysis and 120 patients were included in the expansion analysis. The study protocol was approved by the Institutional Review Board of Asan Medical Center in Seoul based on the signing of the informed consent form (No. 2020-1173). The study was performed in accordance with the Declaration of Helsinki.

### 2. Measurement of SORD

SORD was measured with serum samples collected from the Bio-Resource Center at Asan Medical Center in Seoul. Serum samples were stored in a fresh-frozen state at −196° C. After thawing the samples, sufficient pre-culture time of 24 hours was consumed before initiating the enzymatic reaction. This increased the accuracy in the measurement because the metabolites of the serum, especially ketones, may react with the SORD in the serum. Baseline serum levels of SORD were measured using the Human Sorbitol dehydrogenase ELISA kit (MyBioSource, San Diego, CA, USA) according to the manufacturer's instructions.

### 3. Risk Factors and Outcomes

Clinical pathology data, including preliminary assessment of liver function by Child-Pugh classification, albumin-bilirubin grade (ALBI grade), and indocyanine green retention rate at 15 minutes (ICG-R15) were collected. Hepatectomy was classified according to the Couinaud classification as major resection if more than 4 segments were resected, and as minor resection if less than 4 segments were resected. Tumor size was defined as a diameter of the largest tumor in the surgical specimen.

The outcome of interest was recurrence-free survival (RFS). RFS was defined as a time interval between the date of surgery and the date of recurrence or death. Dynamic computed tomography or magnetic resonance imaging was performed with tumor marker measurements including serum α-fetoprotein (AFP) and protein induced by vitamin K absence or antagonist-II (PIVKA-II) (1 month after resection, at 3-month intervals for the first 2 years thereafter, and every 3 to 6 months in subsequent years). All patients were tracked from the date of surgery until the date of tumor recurrence or death, or until 31 Dec. 2020.

### 4. Statistical Analysis

Technical statistics were represented as median (quartile ranges and numbers with percentages for each of the continuous and categorical variables). Continuous variables were compared using the Mann-Whitney U test. Categorical variables were compared using Fisher's exact test or chi-square test as appropriate. The survival curve for time to event was determined using the Kaplan-Meier analysis and compared using a log-rank test according to the preoperative SORD level. The hazard ratio (HR) and 95% confidence interval (CI) for RFS were calculated using the Cox proportional risk model. Spearman's rank correlation coefficient was estimated between preoperative SORD levels and other prognostic factors for RFS. Statistical analysis was performed using R statistical software version 3.5.0 (R Foundation for Statistical Computing, Vienna, Austria; http://cran.r-project.org/). All tests were bilateral, with p<0.05 considered statistically significant.

### II. Results

### 1. Basic Characteristics of Patients

A total of 92 patients who underwent curative hepatectomy for hepatocellular carcinoma were included in the study. Their median age was 55.0 years, and most were male (76/92, 82.6%), wherein the cause of HCC was chronic hepatitis B (82/92, 89.1%), and they had a Child-Pugh score of 5 (80/92, 87.0%) and underwent minor resection (74/92, 80.4%). Other demographics, including liver function characteristics, treatment methods, and clinicopathological factors, are shown in Table 1. When patients were divided into two groups according to preoperative serum SORD levels, 73 had a preoperative SORD of less than 15 ng/mL and 19 had a preoperative SORD greater than or equal to 15 ng/mL. All baseline characteristics were similar between the two groups, except for the type of resection. Patients with preoperative SORD greater than or equal to 15 ng/mL underwent major resection more frequently than patients with preoperative SORD less than 15 ng/mL. These basic characteristics of the study population are shown in Table 1 below.

TABLE 1

| | Total | | SORD <15 ng/mL | | SORD ≥15 ng/mL | | p- |
|---|---|---|---|---|---|---|---|
| | [N = 92] | | [N = 73] | | [N = 19] | | value |
| Age, median [IQR], y | 55.0 | [47.8, 60.3] | 54.0 | [46.0, 61.0] | 57.0 | [51.0, 59.5] | 0.396 |
| Male, n (%) | 76 | (82.6) | 58 | (79.5) | 18 | (94.7) | 0.220 |
| Etiology, n (%) | | | | | | | 0.791 |
| Hepatitis B | 82 | (89.1) | 65 | (89.0) | 17 | (89.5) | |
| Hepatitis C | 3 | (3.3) | 2 | (2.7) | 1 | (5.3) | |
| Others | 7 | (7.6) | 6 | (8.2) | 1 | (5.3) | |
| Diabetes, n (%) | 19 | (20.7) | 12 | (16.4) | 7 | (36.8) | 0.101 |
| Previous TACE, n (%) | 15 | (16.3) | 11 | (15.1) | 4 | (21.1) | 0.779 |
| AFP, ng/mL | 32.1 | [6.3, 300.5] | 51.4 | [6.4, 270.0] | 11.6 | [7.0, 1501.5] | 0.839 |
| <400, n (%) | 70 | (76.1) | 57 | (78.1) | 14 | (68.4) | 0.564 |
| ≥400, n (%) | 22 | (23.9) | 16 | (21.9) | 6 | (31.6) | 0.564 |
| PIVKA-II, mAU/mL | 45.0 | [21.8, 366.5] | 45.0 | [23.0, 309.0] | 68.0 | [19.0, 495.5] | 0.912 |
| <40, n (%) | 43 | (46.7) | 34 | (46.6) | 9 | (47.4) | 1.000 |
| ≥40, n (%) | 49 | (53.3) | 39 | (53.4) | 10 | (52.6) | 1.000 |
| AST, median [IQR], IU/L | 31.5 | [26.8, 44.0] | 31.0 | [26.0, 42.0] | 39.0 | [28.0, 56.0] | 0.180 |
| ALT, median [IQR], IU/L | 33.0 | [24.0, 44.5] | 32.0 | [24.0, 43.0] | 38.0 | [28.0, 53.0] | 0.243 |
| Platelet, median [IQR], ×10 [symbol] /μL | 131.50 | [110.8, 175.5] | 131.0 | [113.0, 178.0] | 132.0 | [102.0, 159.5] | 0.534 |
| Albumin, median [IQR], g/dL | 3.9 | [3.6, 4.2] | 3.9 | [3.6, 4.2] | 3.7 | [3.5, 4.2] | 0.319 |
| Bilirubin, median [IQR], IU/L | 0.9 | [0.7, 1.1] | 0.9 | [0.7, 1.1] | 0.9 | [0.7, 1.2] | 0.892 |
| Creatinine, median [IQR], mg/dL | 0.8 | [0.7, 0.9] | 0.8 | [0.7, 0.9] | 0.8 | [0.8, 1.0] | 0.635 |
| Prothrombin time, median [IQR], | 1.06 | [1.01, 1.13] | 1.06 | [1.01, 1.13] | 1.06 | [1.01, 1.14] | 0.599 |
| INR | | | | | | | |
| Child-Pugh score, n (%) | | | | | | | 0.666 |
| 5 | 80 | (87.0) | 63 | (86.3) | 17 | (89.5) | |
| 6 | 9 | (9.8) | 8 | (11.0) | 1 | (5.3) | |
| 7 | 3 | (3.3) | 2 | (2.7) | 1 | (5.3) | |
| ALBI grade, n (%) | | | | | | | 0.556 |
| 1 | 38 | (41.3) | 31 | (42.5) | 7 | (36.8) | |
| 2 | 52 | (56.5) | 41 | (56.2) | 11 | (57.9) | |
| 3 | 2 | (2.2) | 1 | (1.4) | 1 | (5.3) | |
| ICG-R15, median [IQR], % | 11.2 | [8.2, 13.8] | 11.0 | [8.2, 13.4] | 13.6 | [8.9, 16.8] | 0.230 |
| Resection type*, n (%) | | | | | | | 0.014 |
| Major | 18 | (19.6) | 10 | (13.7) | 8 | (42.1) | |
| Minor | 74 | (80.4) | 63 | (86.3) | 11 | (57.9) | |
| Tumor size, median [IQR], cm | 3.0 | [2.3, 4.2] | 3.0 | [2.1, 4.3] | 3.3 | [2.8, 3.5] | 0.322 |
| Microscopic vascular invasion, n | 24 | (26.1) | 16 | (21.9) | 8 | (42.1) | 0.136 |
| (%) | | | | | | | |
| Edmondson grade, n (%) | | | | | | | 1.000 |
| I or II | 19 | (20.7) | 15 | (20.5) | 4 | (21.1) | |
| III or IV | 73 | (79.3) | 58 | (79.5) | 15 | (78.9) | |
| Recurrence, n (%) | 43 | (46.7) | 29 | (39.7) | 14 | (73.7) | 0.017 |
| Follow up, median [IQR], month | 57.1 | [19.8, 102.6] | 66.3 | [25.7, 104.3] | 19.7 | [5.3, 44.1] | 0.003 |

Continuous variables following a non-normal distribution were reported as the median (interquartile range [IQR]). Categorical variables were reported as numbers with a percentage (%).
*Major resection was defined as the removal of four or more liver segments, and the rest were considered as minor resections.
AFP, α-fetoprotein; ALBI grade, albumin-bilirubin grade; AST, aspartate aminotransferase; ALT, alanine aminotransferase; ICG-R15, indocyanine green retention rate at 15 minutes; INR, International Normalized Ratio; IU, International Unit; IQR, interquartile range; PIVKA-II, protein induced by vitamin K absence or antagonist-II; SORD, sorbitol dehydrogenase; TACE, Transcatheter arterial chemoembolization. [symbol]
indicates data missing or illegible when filed

2. Recurrence in Accordance with SORD Levels

Recurrence was observed in 43 patients with a median follow-up of 57.1 months. There was a significant difference between people with and without recurrence, with median serum SORD levels of 10.0 ng/mL and 7.1 ng/mL, respectively. In patients with and without recurrence, there were no significant differences in tumor factors such as other basic characteristics including liver function (Child-Pugh score, albumin-bilirubin (ALBI) grade, indocyanine green (ICG) removal rate), size, microscopic vascular invasion, and Edmondson's grade.

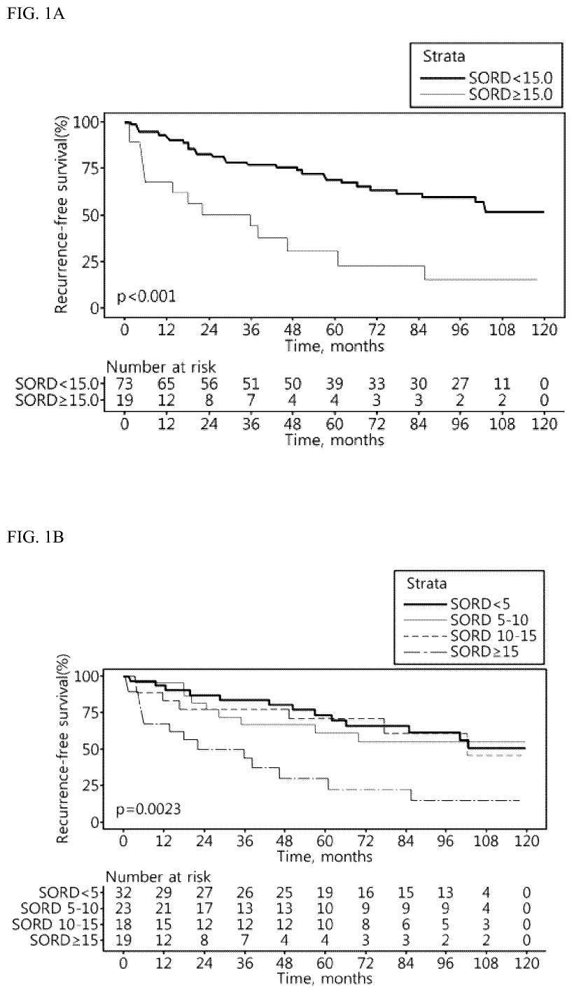

Shown in FIG. 1 are Kaplan-Meier estimates of recurrence-free survival (RFS) obtained by stratifying, by serum SORD levels, Kaplan-Meier plot for recurrence-free survival (RFS) in patients with hepatocellular carcinoma after curative resection. FIG. 1A shows patients stratified into two groups according to preoperative serum SORD levels (<15 ng/mL and ≥15 ng/mL), and FIG. 1B shows patients stratified into four groups according to preoperative serum SORD levels (<5, 5-10, 10-15, ≥15 ng/mL). Overall, the median post-curative resection and 2-year RFS rates of HCC were 100.3 months and 76.3%, respectively. When subjects were stratified into two groups (≥15 ng/mL versus <15 ng/mL) according to preoperative serum SORD levels, the group with the higher levels had a worse outcome with a 2-year RFS rate of 50.1%, compared to RFS rates of 83.0% (p<0.001) for those with low serum SORD levels (FIG. 1A). When patients were divided into four groups according to preoperative serum SORD levels (<5, 5-10, 10-15, ≥15 ng/mL), RFS was similar in patients with levels of <15 ng/mL (FIG. 1B). However, RFS was significantly low in patients with SORD levels of ≥15 ng/mL compared to the other groups.

When patients with Barcelona Liver Cancer (BCLC) stage B or C were included in the expansion analysis, the results were similar to those of the original analysis with worse outcomes in patients with baseline serum SORD levels of ≥15 ng/mL.

3. Prognostic Factors Associated with Recurrence-Free Survival

Univariate and multivariate Cox proportional risk regression analysis was performed to investigate prognostic factors for RFS after curative hepatectomy (see Table 2 below).

TABLE 2

| | Univariate analysis | | | Multivariate analysis | | |
|---|---|---|---|---|---|---|
| Variable | HR | 95% CI | p-value | HR | 95% CI | p-value |
| Age ≥60 y | 1.02 | 0.52-1.98 | 0.96 | — | — | — |
| Male | 2.57 | 0.92-7.18 | 0.073 | 1.83 | 0.64-5.21 | 0.261 |
| ICG R15 ≥10% | 1.71 | 0.90-3.36 | 0.099 | 1.77 | 0.93-3.38 | 0.083 |
| Child-Pugh score ≥6 | 0.77 | 0.30-1.97 | 0.591 | — | — | — |
| ALBI grade ≥2 | 1.37 | 0.74-2.54 | 0.310 | — | — | — |
| Tumor size ≥5 cm | 0.98 | 0.44-2.21 | 0.966 | — | — | — |
| Microscopic vascular | 1.48 | 0.77-2.83 | 0.242 | — | — | — |
| invasion | | | | | | |
| AFP ≥400 ng/mL | 1.68 | 0.86-3.27 | 0.129 | 2.03 | 1.01-4.07 | 0.047 |
| PIVKA-II ≥40 mAU/mL | 1.24 | 0.68-2.27 | 0.479 | — | — | — |
| SORD ≥15 ng/mL | 3.29 | 1.72-6.28 | <0.001* | 3.46 | 1.76-6.81 | <0.001 |
| Major hepatectomy* | 1.63 | 0.80-3.30 | 0.18 | — | — | — |

*Major resection was defined as the removal of four or more liver segments.
AFP, α-fetoprotein; ALBI grade, albumin-bilirubin grade; CI, confidence intervals; HR, Hazard ratio; ICG-R15, indocyanine green retention rate at 15 minutes; PIVKA-II, protein induced by vitamin K absence or antagonist-II; SORD, sorbitol dehydrogenase.

In the multivariate regression analysis, high serum α-fetoprotein (AFP) levels (≥400 ng/mL; hazard ratio (HR), 2.03, 95% confidence interval (CI, 1.01-4.07, p=0.047)) and high serum SORD levels (≥15 ng/mL; HR, 3.46, 95% CI 1.76 to 6.81, p<0.001) were identified as an independent prognostic factor for RFS (see Table 2).

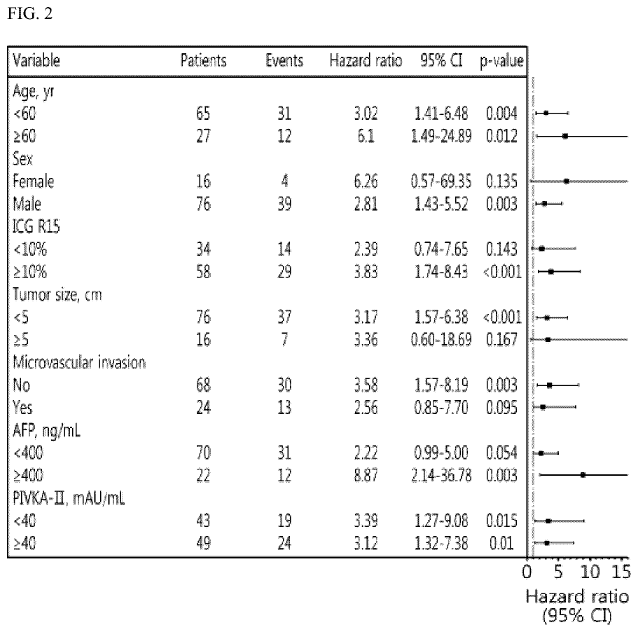

The forest plot of recurrence-free survival by preoperative serum SORD levels in the subgroup of patients is shown in FIG. 2, which refers to the hazard ratio for RFS in patients with high serum SORD levels (≥15 ng/mL) compared to patients with low serum SORD levels (<15 ng/mL). When subgroup analysis was performed, patients with high preoperative SORD levels were found to have worse RFS in all subgroups compared to patients with low preoperative SORD levels (see FIG. 2).

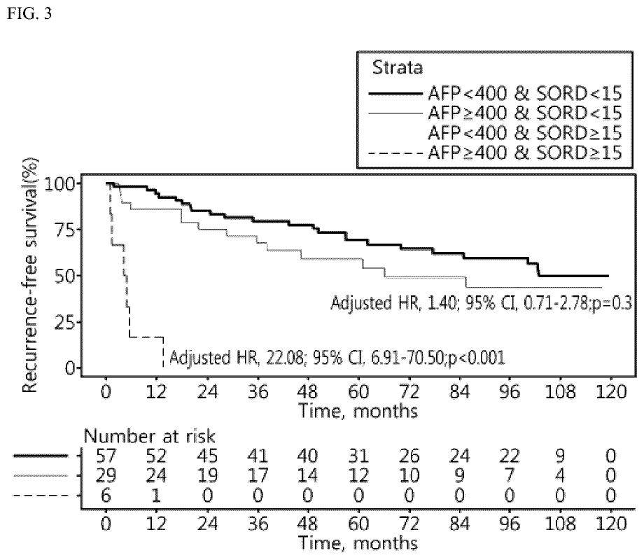

For reference, the effect size of preoperative SORD levels for RFS was high in patients with high serum AFP levels (≥400 ng/mL; HR, 8.87; 95% CI, 2.14-36.78; p=0.003) compared to patients with low serum AFP levels (<400 ng/mL; HR, 2.22; 95% CI, 0.99-5.00; p=0.054). This suggests that preoperative serum SORD levels are particularly good predictors of outcomes in patients with high serum AFP levels, and further stratify patients at high risk of recurrence along with serum AFP levels. Based on these results, when patients were stratified by preoperative serum SORD and AFP, patients with elevated both AFP and SORD levels had the lowest RFS rate and a markedly poor prognosis (HR, 22.08; 95% CI, 6.91 to 70.50; p<0.001)). The RFS rates in the other two groups were found to be similar (HR, 1.40; 95% CI, 0.71-2.78; p=0.30) (see FIG. 3).

4. Factors Correlated with Serum SORD

Spearman's rank correlation coefficients were calculated to examine factors correlated with SORD levels. SORD levels were positively correlated with indocyanine green retention rate at 15 minutes (ICG-R15) (r=0.27, p=0.008) and AST (r=0.23, p=0.027) and negatively correlated with albumin (r=−0.26) (r=−0.26; p=0.011). There was no correlation between SORD levels and other tumor markers, including AFP (r=0.002, p=0.99) and proteins induced by vitamin K absence or antagonist-II (PIVKA-II) (r=0.08, p=0.46).

When assessing the correlation between the pathological characteristics of HCC and preoperative serum SORD levels, microvascular invasion or the presence of Edmonson-Steiner grade were not associated with preoperative SORD levels. However, subjects with higher ALBI grades tended to have higher serum SORD levels. When patients with BCLC stage B or C were included in the expansion analysis, patients with higher ALBI grades or higher Child-Pugh scores were found to have higher preoperative serum SORD levels. This indicates that baseline preoperative SORD levels may reflect liver functions.

### III. Discussion

This study evaluated the association between preoperative serum SORD levels and surgical outcomes in patients who underwent curative resection for hepatocellular carcinoma. Compared to a case in which patients with low preoperative serum SORD levels (<15 ng/mL) had 2-year RFS of 83.0%, patients with high preoperative serum SORD levels (≥15 ng/mL) had a significantly worse 2-year prognosis of 50.1%. In multivariate Cox regression analysis, high SORD levels were identified as a statistically significant prognostic factor associated with RFS after curative resection for HCC. For reference, in subgroup analysis, preoperative SORD levels were particularly good predictors of surgical outcomes in patients with high serum AFP levels (≥400 ng/mL) compared to patients with low serum AFP levels (<400 ng/mL). When patients were stratified by preoperative serum AFP and SORD levels, patients with elevated preoperative levels of both AFP and SORD had a markedly poor prognosis.

SORD is an enzyme found primarily in the liver, although it is present in many tissues of the body. In general, serum SORD levels are low, but in cases of liver damage, levels increase with ALT and AST. This suggests that elevated SORD levels indicate hepatocyte damage. Chronic hepatocyte damage and hepatocyte necrosis cause myofibroblast activation, leading to liver fibrosis and cirrhosis. In cirrhosis, persistent hepatocyte damage contributes to carcinogenesis by destroying telomeres, releasing reactive oxygen species, and altering paracrine signaling in the cellular microenvironment. In addition, previous proteomic studies have revealed that lower SORD expression in liver tissue in patients with hepatocellular carcinoma results in low survival (Human Protein Atlas. Availabe online: https://www.proteinatlas.org/ENSG00000140263-SORD/pathology/liver+cancer (accessed on 26 July).

When SORD activity is low or absent in the liver, sorbitol may accumulate in hepatocytes. If sugar alcohols, including sorbitol, accumulate in the liver due to a lack of SORD activity, they may contribute to the development of liver cancer. In addition, if the lack of SORD activity activates the corresponding pathways, such as the polyol pathway in cancer cells, it may contribute to the further accumulation of sorbitol, which may lead to promotion of poor differentiation of HCCs.

In the present disclosure, high serum AFP levels (2400 ng/mL) and high serum SORD levels (≥15 ng/mL) were identified as two independent prognostic factors for RFS based on multivariate Cox regression analysis, but the effect size of SORD for predicting surgical outcome in this study was greater than that of AFP. Interestingly, the interaction (subgroup) analysis showed that SORD and AFP interact in predicting surgical outcomes and that the predictive ability of SORD improves in patients with elevated AFP levels. These results suggest that SORD may complement the prognostic ability of AFP in hepatocellular carcinoma patients who had undergone with HCC resection for complete cure. In fact, patients with elevated levels of both SORD and AFP had a severe prognosis in a median RFS of less than six months.

Other well-known prognostic factors for recurrence of hepatocellular carcinoma after curative resection include microvascular invasion and tumor size. However, in the current study, these histological factors were not significantly associated with RFS. This may be due to the characteristics of patients who are in BCLC stage 0 or A with a small tumor (median, 3.0 cm). Patients with advanced BCLC stage and tumor in a large size were not included in this study because resection for complete cure is not the standard of care. Previous studies have noted that microvascular invasion does not affect long-term surgical outcomes, especially in patients with early-stage hepatocellular carcinoma.

To our knowledge, no previous studies have examined the correlation between serum SORD levels and other prognostic factors for hepatocellular carcinoma. There was no significant association between SORD levels and tumor prognostic factors such as tumor size, AFP, and PIVKA-II in this study. However, SORD levels were positively associated with AST levels, ICG-R15 and ALBI grades, which reflect liver damage or liver function. SORD levels have the advantage in prediction of the prognosis and reflection of liver function in comparison with other conventional tumor markers, such as AFP and PIVKA-II. However, further study is needed to determine the effectiveness of SORD levels in assessing liver function. Nonetheless, since it is easier to obtain blood samples, unlike tissue samples, SORD may be utilized as a more efficient and useful prognostic predictor in clinical practice in HCC patients.

In conclusion, elevated levels of preoperative serum SORD (≥15 ng/mL) were significantly associated with the poor prognosis in patients with hepatocellular carcinoma after curative resection. Moreover, preoperative serum SORD and AFP levels were better predictive of outcomes when used together and were likely to predict a poor prognosis particularly in patients with elevated levels of SORD (≥15 ng/mL) and AFP (≥400 ng/mL). Based on these results, it is expected that the use of serum SORD alone or in combination with AFP levels would facilitate decision-making in selecting patients for whom surgical treatment is appropriate in the clinical field and avoid unnecessary surgery in patients who are expected to have low postoperative survival.

1. A method of diagnosing a prognosis after liver cancer surgery, comprising:
contacting a blood sample obtained from a liver cancer patient prior to the liver cancer surgery with an antibody specifically binding to an L-iditol-2 dehydrogenase (SORD) protein;
detecting a level of the SORD protein in the blood sample;
determining whether the level of the SORD protein in greater than or equal to 15 ng/mL; and
diagnosing a poor prognosis after the liver cancer surgery if the the level of the SORD protein in greater than or equal to 15 ng/mL.

2. The method of claim 1, wherein the prognosis is predicted prior to liver cancer surgery.

3. The method of claim 1, wherein the blood sample is serum.

4-5. (canceled)

6. The method of claim 3, wherein the method further comprises:
detecting a level of alpha-fetoprotein (AFP) by contacting the serum obtained from the liver cancer patient prior to the liver cancer surgery with an antibody specifically binding to the AFP; and
determining whether the serum AFP level is greater than or equal to 400 ng/mL.

7. The method of claim 6, wherein, when the serum SORD level is greater than or equal to 15 ng/mL and the serum AFP level is greater than or equal to 400 ng/mL, information for predicting a poor prognosis after liver cancer surgery is provided.

8. The method of claim 6, wherein detection of the SORD or the AFP level is performed by immunoassay or immunostaining.

9. The method of claim 8, wherein the immunoassay or immunostaining is performed using the antibody specifically binding to the SORD or the AFP.

10. A method of early diagnosing a prognosis of liver cancer surgery to detect a SORD marker from a blood sample, comprising:
contacting the blood sample obtained from a liver cancer patient prior to the liver cancer surgery with a compositing comprising an antibody specifically binding to an L-iditol-2 dehydrogenase (SORD) protein;
detecting a level of the SORD protein in the blood sample;
determining whether the level of the SORD protein in greater than or equal to 15 ng/mL; and
diagnosing a poor prognosis after the liver cancer surgery if the the level of the SORD protein in greater than or equal to 15 ng/mL.

11. The method of claim 10, wherein the composition further comprises an antibody specifically binding to an alpha-fetoprotein (AFP) protein.

12. The method of claim 10, wherein the composition is a composition for detecting a serum protein.

13. A kit for early diagnosing a prognosis of liver cancer surgery to detect a SORD marker from a blood sample, comprising:
a manual that instructs to provide information for predicting a poor prognosis after liver cancer surgery based on the method of claim 1.

14. The kit of claim 13, wherein the manual that instructs to provide the information for predicting the poor prognosis after the liver cancer surgery based on the method of claim 6.

15. (canceled)
